(19) United States
(12) Patent Application Publication
(10) Pub. No.: US 2026/0345230 A1
(43) Pub. Date: Nov. 5, 2026

(54) Water Vapor Generation System with Improved Efficiency and Control

(71) Applicant: Virender K. Sharma, Paradise Valley, AZ (US)

(72) Inventor: Virender K. Sharma, Paradise Valley, AZ (US)

(21) Appl. No.: 19/562,027

(22) Filed: Mar. 10, 2026

Related U.S. Application Data
(63) Continuation-in-part of application No. 19/349,912, filed on Oct. 3, 2025.
(60) Provisional application No. 63/703,817, filed on Oct. 4, 2024.
(60) Provisional application No. 63/741,921, filed on Jan. 5, 2025.
(60) Provisional application No. 63/769,517, filed on Mar. 10, 2025.

(51) Int. Cl. A61B 18/14; A61B 18/00

(52) U.S. Cl. CPC A61B 18/1492; A61B 2018/00577; A61B 2018/00625; A61B 2018/00642

(57) ABSTRACT

A catheter-based vapor generation system includes a fluid delivery lumen having a plurality of ports, an electrode positioned proximate the fluid delivery lumen, and a porous vapor-permeable and liquid-impermeable or liquid-restrictive membrane proximate the saline delivery lumen and the electrode. The porous membrane is configured to permit passage of vapor while restricting passage of bulk liquid. An impedance-based energy control system monitors electrode impedance and controls energy delivery to the electrode for optimized vapor generation.

### CROSS-REFERENCE TO RELATED APPLICATIONS

The present application relies on U.S. Patent Provisional Application No. 63/769,517, titled “Water Vapor Generation System with Improved Efficiency and Control” and filed on Mar. 10, 2025, for priority, which is herein incorporated by reference in its entirety.

The present application is also a continuation-in-part application of U.S. patent application Ser. No. 19/349,912, titled “Balloon Catheter Ablation Systems and Methods for Patterned Tissue Ablation” and filed on Oct. 3, 2025, which relies on U.S. Patent Provisional Application No. 63/741,921, titled “Methods and Devices for Endovascular Treatment” and filed on Jan. 5, 2025 and U.S. Patent Provisional Application No. 63/703,817, titled “Heated Vapor Ablation Systems and Methods” and filed on Oct. 4, 2024, for priority, all of which are incorporated herein by reference in their entirety.

### FIELD

The present invention relates to a radiofrequency (RF) vapor generation system, more specifically, to an improved catheter-based system that enhances vapor quality, energy efficiency, and delivery control for medical applications.

### BACKGROUND

Radiofrequency-based vapor generation has been widely utilized in medical procedures such as tissue ablation and vessel treatment. Prior art devices predominantly rely on a linear RF heating process where a saline solution flows over an RF electrode, undergoing a phase transition into vapor before being delivered through distal ports in an inline arrangement. In this design, the saline moves continuously from its source, over the RF heating electrode, and toward the vapor delivery ports, following a direct path. However, this method presents several challenges that affect efficiency and performance.

One of the primary issues associated with prior art devices is condensation and blockage. As the vapor moves through the RF heating chamber toward the delivery ports, cooling causes condensation, which can obstruct some or all of the ports. This obstruction reduces the efficiency and uniformity of vapor delivery, as traditional vapor delivery ports have diameters exceeding 0.1 mm. Additionally, energy loss and irregular delivery arise due to the presence of liquid water in the vapor output. Expelling a mixture of steam and liquid water results in wet steam, which creates inconsistent heating of the tissue and diminishes the overall effectiveness of the treatment.

Another significant limitation of existing RF vapor generation systems is salinity buildup and impedance variations. As water vaporizes, the remaining saline solution becomes increasingly concentrated, raising its salinity and altering the impedance characteristics of the RF electrode. This impedance shift affects the efficiency of RF energy delivery, leading to erratic vaporization rates and unpredictable energy transmission to the target tissue. Furthermore, the inability to dynamically control RF energy output in response to these variations results in inefficient vapor generation and inconsistent therapeutic outcomes.

Most of the current systems and methods of ablation suffer from limitations such as condensation, port blockage, wet vapor output with variable vapor quality, and efficiency loss making dosimetry complex and variable. Further, as water vaporizes from an ionic working fluid, solute concentration may increase, which can change electrical load characteristics (such as impedance) and destabilize heating, leading to inconsistent vapor generation and therapeutic delivery. Given these challenges, there is a pressing need for an RF vapor generation system that optimizes vapor purity, enhances energy efficiency, and prevents blockages in the vapor delivery process.

### SUMMARY

The following embodiments and aspects thereof are described and illustrated in conjunction with systems, tools and methods, which are meant to be exemplary and illustrative, and not limiting in scope. The present application discloses numerous embodiments.

The present specification is directed to a catheter-based vapor generation system comprising: an elongate shaft comprising a fluid delivery lumen longitudinally extending within the elongate shaft, wherein the elongate shaft has a distal end; at least one electrode positioned proximate to an end of the fluid delivery lumen at the distal end of the elongate shaft and configured to heat a working fluid delivered by the fluid delivery lumen to generate a heated vapor-rich two-phase mixture; a porous membrane filter completely surrounding the at least one electrode and defining an enclosed annular volume, wherein the porous membrane filter is configured to permit passage of the vapor from the two-phase mixture out of said enclosed annular volume while restricting a release of liquid from the two-phase mixture, thereby maintaining liquid within said enclosed annular volume; an impedance sensing module configured to measure an electrode-fluid load impedance associated with the at least one electrode; and a controller configured to: control delivery of energy to the at least one electrode; and modify at least one operating parameter of the vapor generation system in response to a detected change in the electrode-fluid load impedance.

Optionally, the energy is radio frequency (RF) and the at least one operating parameter is at least one of RF power, RF frequency, RF duty cycle, a rate of the working fluid flow, and a volume of the working fluid flow.

Optionally, the porous membrane filter comprises a hydrophobic polymer material adapted to retain said liquid within the annular volume while permitting said vapor to flow out said annular volume. Optionally, the porous membrane filter comprises pore sizes having a size range from 0.1 micrometer (μm) to 1.0 μm.

Optionally, the energy is radio frequency (RF) energy and the controller comprises an adaptive RF modulation system that adjusts at least one of RF power and RF frequency based on measured impedance changes at the at least one electrode.

Optionally, the annular volume is defined between an inner surface of the porous membrane filter and at least one of an outer surface of the fluid delivery lumen and an outer surface of the at least one electrode.

Optionally, the annular volume comprises at least one structural support member disposed between the at least one electrode and the porous membrane filter and configured to maintain a predetermined working fluid layer thickness.

Optionally, the at least one electrode comprises a plurality of traces separated from each other by a spacing and the at least one structural support extends perpendicularly from a surface of the at least one electrode and has a height equal to about 0.2 to 10 times the inter-trace distance.

Optionally, the controller is configured to trigger a fluid flush cycle in response to a detected impedance of liquid in the annular volume corresponding to an increased salinity of the working fluid. Optionally, the increased salinity is defined by a saline concentration of the liquid in the annular volume exceeding 5%. Optionally, the system further comprises a fluid return lumen in fluid communication with the annular volume and configured to remove liquid from the annular volume during the fluid flush cycle. Optionally, the system further comprises a gas delivery lumen, separate from the fluid delivery and the fluid return lumen, configured to introduce a gas into the annular volume such that the gas mixes with generated vapor to form a gas-vapor mixture delivered through the porous membrane filter. Optionally, the gas comprises carbon dioxide and forms a heated gas-vapor mixture with the generated vapor.

The present specification also discloses a method of generating and delivering heated vapor, comprising: providing a catheter having a distal end comprising at least one electrode, a porous filter circumferentially positioned around the at least one electrode and defining a vaporization region, and a fluid delivery lumen having an end terminating proximate the at least one electrode and the porous filter; delivering a working fluid through the fluid delivery lumen and into the vaporization region; applying RF energy to the at least one electrode in the vaporization region to generate a two-phase mixture of vapor and liquid; permitting the vapor of the two-phase mixture to pass through the porous membrane and out of the vaporization region while concurrently restricting the liquid of the two-phase mixture to pass through the porous membrane and out of the vaporization region; measuring an electrode-fluid load impedance associated with the at least one electrode; using a controller, monitoring an electrical parameter indicative of a change in working fluid concentration based on the measured electrode-fluid load impedance; using the controller, adjusting at least one of RF power, RF duty cycle, RF frequency, and working fluid flow responsive to the monitored electrical parameter; and performing a flush cycle by introducing fresh working fluid into the vaporization region through the fluid delivery lumen and removing concentrated working fluid in the vaporization region through a return lumen.

Optionally, the porous membrane comprises a hydrophobic polymer material adapted to retain said liquid within the vaporization region while permitting said vapor to flow out said vaporization region and comprises pore sizes having a size range from 0.1 micrometer (μm) to 1.0 μm.

Optionally, the vaporization region comprises at least one structural support member disposed between the at least one electrode and the porous membrane and configured to maintain a predetermined working fluid layer thickness. Optionally, the working fluid layer thickness is equal to about 0.2 to 10 times an inter-trace distance of the at least one electrode. Optionally, the at least one electrode comprises a plurality of traces separated from each other by a spacing and wherein the at least one structural support extends perpendicularly from a surface of the at least one electrode and has a height equal to about 0.2 to 10 times the inter-trace distance. Optionally, the vaporization region has a thickness equal to about 0.2 to 10 times an inter-trace distance of the at least one electrode.

Optionally, the catheter further comprises a gas delivery lumen, separate from the fluid delivery and the return lumen, configured to introduce a gas into the volume such that the gas mixes with generated vapor to form a gas-vapor mixture delivered through the porous membrane.

In some embodiments, the present specification is also directed toward a catheter-based RF vapor generation system comprising: a saline delivery lumen with a plurality of ports; an RF electrode positioned proximate to the saline delivery lumen; a porous membrane vapor outlet surrounding the saline delivery lumen and the RF electrode, the porous membrane configured to permit passage of vapor while restricting passage of bulk liquid; and, an impedance-based RF energy control system that monitors electrode impedance.

Optionally, the impedance-based RF energy control system is configured to programmatically adjust RF delivery or saline flow.

Optionally, the porous membrane is composed of a hydrophobic material capable of retaining liquid condensate while permitting gaseous vapor flow.

Optionally, the system further comprises an adaptive RF modulation mechanism that adjusts power or frequency based on measured impedance changes due to saline concentration variations.

Optionally, the impedance-based control mechanism triggers a saline flush cycle upon detecting a threshold concentration exceeding 5%.

Optionally, the present specification is directed towards a method for addressing a change in electrode impedance within a RF vapor generation system comprising: heating a saline solution within a saline delivery lumen using RF energy; detecting a change in electrode impedance; and, adjusting at least one of an RF output or a saline flow to modify the vapor generation.

Optionally, the present specification is directed towards a method for addressing a change in electrode impedance within a RF vapor generation system comprising: heating a saline solution within a saline delivery lumen using RF energy; detecting a change in electrode impedance; measuring an electrode impedance value that is indicative of a salinity of the saline solution; determining if the salinity of the saline solution has reached a predetermined threshold value based on said electrode impedance value; initiating a generator flush cycle if the salinity of the saline solution is greater than the predetermined threshold value; and, stopping the generator flush cycle when the salinity of the saline solution is at or below said predetermined threshold value based on another electrode impedance measurement.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1 is a diagram of an RF vapor generation system of the present specification, in some embodiments, illustrating the coaxial arrangement of the saline lumen, RF electrode, and porous membrane;

FIG. 2A illustrates a longitudinal cross-sectional view of catheter, in accordance with some embodiments of the present specification;

FIG. 2B illustrates a lateral cross-sectional view of a distal portion of catheter comprising the RF vapor generation system in accordance with some embodiments of the present specification;

FIG. 2C illustrates an exemplary design of an electrode configuration, in accordance with some embodiments of the present specification;

FIG. 2D separately illustrates the different layers used to form the electrode configuration of FIG. 2C, in accordance with some embodiments of the present specification;

FIG. 3A is a schematic view of an example flexible RF heater strip having a plurality of electrodes positioned adjacent a porous membrane outlet, and further illustrating offsets that define a gap between the electrodes and the porous membrane outlet;

FIG. 3B is a schematic view illustrating introduction of a working fluid into the chamber between the flexible RF heater strip and the porous membrane outlet to form a working-fluid layer;

FIG. 3C is a schematic view illustrating delivery of RF energy through the working-fluid layer to generate vapor and expel vapor through the porous membrane outlet;

FIG. 4A illustrates a longitudinal cross-section view of catheter with a gas delivery lumen, similar to FIG. 2A, in accordance with some embodiments of the present specification;

FIG. 4B illustrates a lateral cross-section view of a distal section of catheter with a gas delivery lumen, similar to FIG. 2B, in accordance with some embodiments of the present specification;

FIG. 5A illustrates an electrical and control architecture for a catheter-based RF vapor generation system configured to improve vapor quality, heating efficiency, and controllability using electrical-parameter sensing and closed-loop control, in accordance with some embodiments of the present specification;

FIG. 5B illustrates a fluidics architecture for delivering working fluid to a catheter vaporization region and selectively directing return fluid to waste or to a recirculation loop, under the control of controller;

FIG. 6 is a flow diagram illustrating a method of impedance change management by the generator in response to increasing saline concentration in the vaporization chamber;

FIG. 7 is a flow diagram showing a method of saline level management by the generator once a threshold saline concentration is reached for continuous and efficient vapor generation;

FIG. 8A is a timing diagram illustrating RF power delivered to an electrode/fluid load and a corresponding delivered-energy accumulation profile for repeated treatments;

FIG. 8B is a block diagram illustrating a control architecture for delivering a target energy to an electrode/fluid load using RF power while adapting RF delivery based on an estimated electrical parameter and optionally initiating a flush operation when a threshold condition is met; and

FIG. 9 is a graph illustrating a relationship between electrode-fluid impedance and time to vaporization.

### DETAILED DESCRIPTION

The present specification describes systems, devices, and methods for generating and delivering heated vapor using radiofrequency (RF) energy within a catheter-based platform. The disclosed system improves vapor quality, energy efficiency, and delivery control relative to conventional RF vapor generation devices.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term “vapor generation system” refers to any or all of the heater or induction-based approaches to generating steam from water described in this application.

All ablation catheters disclosed in the specification, in some embodiments, include insulation at the location of the electrode(s) to prevent ablation of tissue proximate the location of the electrode within the catheter.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

Ablative agents are inexpensive and readily available and are directed via an infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor may use temperature, pressure or other sensing data to control the flow of the ablative agent. In addition, one or more suction ports are provided to suction the ablation agent from the vicinity of the targeted tissue. The targeted segment can be treated by a continuous infusion of the ablative agent or via cycles, where each cycle comprises infusion and removal of the ablative agent as determined and controlled by the microprocessor.

In some embodiments, a catheter includes at least one delivery lumen configured to introduce working fluid into a vaporization chamber and at least one return lumen configured to remove liquid from the vaporization chamber and/or a phase-separator chamber using suction. The system may operate in continuous flow, intermittent flow, or cyclic modes, and may execute flush cycles in which fresh working fluid is introduced while concentrated working fluid is exhausted through the return lumen.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

It should be noted herein that any feature or component described in association with a specific embodiment may be used and implemented with any other embodiment unless clearly indicated otherwise.

In general, the present specification describes a catheter-based RF vapor generation system that converts a working fluid, such as saline, into heated vapor within a controlled vaporization chamber and delivers the vapor through a porous membrane outlet while restricting the passage of bulk liquid. The system integrates: a working fluid delivery pathway, one or more RF heating electrodes, a phase-separation membrane outlet, an impedance-aware RF control system, an optional fluid recirculation or flushing architecture, and an optional gas co-delivery architecture. These elements cooperate to produce controlled vapor generation while maintaining consistent electrical loading conditions at the electrode-fluid interface.

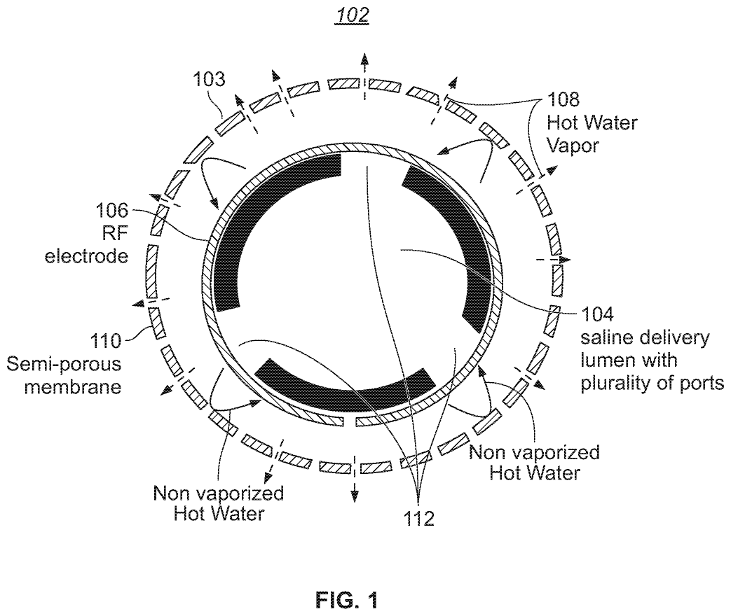

Referring to FIG. 1, a catheter-based vapor generation system is illustrated which includes a coaxial vapor pathway 102. FIG. 1 illustrates a lateral sectional view of a distal catheter portion comprising: coaxial vapor pathway 102, an inner saline delivery lumen 104 through which working fluid is delivered, an RF electrode 106 positioned adjacent to lumen 104, a vaporization region where the working fluid is heated to produce vapor 108, a porous hydrophobic membrane layer (termed herein also as ‘porous membrane vapor filter’) 110 surrounding the vaporization region, and one or more saline exit ports 112. In some embodiments, each port 112 has a diameter ranging from 0.1 to 1.0 mm. In various embodiments, ports 112 are one or more in numbers, and additionally they are circular, oval, or of any other shape. The porous membrane layer may comprise a hydrophobic microporous polymer material, such as: expanded polytetrafluoroethylene (ePTFE), porous PTFE, flash-spun high-density polyethylene, polypropylene, polyethylene, or other vapor-permeable hydrophobic materials. In embodiments, porous membrane vapor filter 110 has a thickness ranging from 5 to 500 microns. In some embodiments, catheter shaft 103 has an outer diameter ranging from 2 to 5 millimeter (mm). In some embodiments, a diameter of fluid delivery lumen 104 ranges from 0.5 to 3 mm. In some embodiments, pores of porous membrane vapor filter 110 have diameters ranging from 0.1-0.45 μm. The membrane permits passage of vapor while restricting bulk liquid water, thereby acting as a phase separator that promotes delivery of relatively dry vapor. In contrast to conventional systems in which saline flows linearly across a heating electrode before exiting through distal ports, the present configuration introduces a coaxial vapor generation structure that promotes efficient heating and controlled phase separation. Furthermore, condensed liquid retained within the chamber remains in thermal communication with the RF electrode 106, allowing reheating and revaporization.

In some embodiments, the porous membrane outlet (also referred to herein as a semi-porous outlet or phase-separator membrane) comprises a hydrophobic, vapor-permeable and liquid-restrictive membrane configured to permit passage of vapor while restricting passage of bulk liquid. In some embodiments, the membrane is characterized by a porosity defined as a void fraction (i.e., percent void volume). The void fraction may be selected based on desired vapor flux, resistance to liquid breakthrough, and pressure drop across the membrane. In some embodiments, the membrane void fraction is in a range from 10% to 98%, and in preferred subranges of 35% to 95% and, more preferably, 50% to 90%. In some embodiments, the membrane comprises a flash-spun polyolefin nonwoven, for example, flash-spun high-density polyethylene (HDPE), a porous PTFE membrane, an expanded PTFE (ePTFE) membrane, and/or an electrospun fluoropolymer membrane. In some embodiments, the void fraction may be selected according to the membrane type, for example: 45% to 85% for flash-spun polyolefin nonwovens, 20% to 90% for porous PTFE membranes, and 50% to 98% for ePTFE and/or electrospun fluoropolymer membranes. In some embodiments, the membrane may further be characterized by one or more additional structural parameters including pore size distribution, membrane thickness, wettability or hydrophobicity, tortuosity, and/or effective open area. Any one or more of these parameters may be selected alone or in combination with the void fraction to achieve desired vapor-permeable and liquid-restrictive behavior.

In some embodiments, the porosity (void fraction) of the porous membrane outlet is selected to control a back pressure within the vapor generation chamber during vapor generation. For example, the porous membrane outlet may function as a flow restrictor such that a pressure drop across the porous membrane outlet is determined by one or more membrane parameters including void fraction, pore size distribution, membrane thickness, tortuosity, and/or outlet area. In some embodiments, selecting a lower void fraction (and/or smaller effective pore size and/or increased thickness) increases flow resistance and increases chamber back pressure for a given vapor generation rate, while selecting a higher void fraction (and/or larger effective pore size and/or decreased thickness) reduces flow resistance and reduces chamber back pressure. In some embodiments, the chamber back pressure is selected within a broad range, for example from 0.8 atm to 5 atm (and preferred subranges such as 1 atm to 3 atm), although higher and lower chamber pressures are contemplated depending on the application.

In some embodiments, increasing chamber back pressure improves one or more of vapor temperature and vapor quality. For example, increasing chamber pressure increases the saturation temperature of water, such that vapor generation can occur at a higher equilibrium temperature than at atmospheric pressure, consistent with the pressure-temperature relationship of phase change. In some embodiments, increased chamber pressure also increases vapor density (consistent with gas-law behavior), which can affect volumetric flow, convective transport, and delivery characteristics of the vapor and/or vapor-gas mixture. In some embodiments, controlling back pressure reduces entrained bulk liquid and promotes a higher quality vapor output by stabilizing boiling and reducing expulsion of liquid slugs through the outlet. In some embodiments, the system selects the membrane porosity and/or other membrane parameters to target a desired chamber pressure and thereby achieve a desired vapor quality and/or temperature profile, including to increase delivered energy density and/or extend thermal transport relative to vapor alone.

In some embodiments, chamber back pressure is monitored directly (e.g., via a pressure sensor) and/or indirectly (e.g., via inferred flow resistance or electrical proxies), and the system adjusts one or more operating parameters to maintain a selected chamber pressure. Non-limiting examples include adjusting RF power delivery, duty cycle, working fluid flow rate, and/or a valve or restrictor in the exhaust path, in combination with (or independent of) selection of membrane porosity.

As shown in FIG. 1, the catheter-based RF vapor generation system described in accordance with some embodiments of this specification incorporates a coaxial vapor pathway 102. The coaxial vapor pathway 102 defines an annular vaporization chamber surrounding inner saline delivery lumen 104. In some embodiments, volume of the chamber ranges from 0.5 milliliters (ml) to 5 ml. This chamber is bounded radially outward by porous membrane vapor filter 110 and radially inward by the outer surface of saline lumen 104 and/or at least one electrode 106. Pathway 102 provides confined vaporization space, enables re-heating of condensed saline, and prevents linear downstream cooling.

In some embodiments, the porous membrane vapor filter 110 completely surrounds the at least one electrode 106. As used herein, the phrase “completely surrounding” is defined in that the porous membrane filter completely encircles electrode 106 about a circumferential direction such that vapor generated within the annular volume must pass through porous membrane vapor filter 110 in order to exit the elongate shaft. In these embodiments, porous membrane vapor filter 110 forms a continuous vapor-permeable boundary around the vaporization region and prevents vapor from escaping directly into the catheter shaft without first passing through porous membrane vapor filter 110.

Saline delivery lumen 104 extends longitudinally within the catheter shaft and is configured to transport saline toward the distal region. One or more exit ports 112 are formed in lumen 104. These ports permit saline to exit radially into vaporization chamber 102 rather than flow axially over the electrode. This radial discharge configuration distinguishes the present specification from prior linear-flow architectures.

RF electrode 106 is positioned within chamber 102 such that saline exiting ports 112 contact electrode 106 directly. Electrode 106 is powered using an RF power source ranging from 20 to 200 Watt (W). RF electrode 106 generates Joule heating through dielectric polarization and ionic conduction. The electrode is electrically coupled to an RF generator (not shown) and operates within a selected frequency range, for example between 1 MHz and 27 MHz. Lower frequencies (1-6 MHz) allow for deeper tissue penetration and broader energy diffusion, while higher frequencies (13-27 MHz) provide more localized heating and precise vaporization control. The intensity of heating is influenced by saline concentration, RF frequency, and electrode configuration, ensuring efficient energy transfer for vaporization. Impedance at RF electrode 106 is monitored by impedance sensors that are in communication with the RF generator. In embodiments, RF electrode 106 comprises one of: a ring, mesh, coil, cylindrical sleeve, or is otherwise configured to provide surface area for heating. In accordance with the present specification, impedance at RF electrode 106 is varied as the salinity increases due to vaporization-induced salt concentration changes.

The RF vaporization process takes advantage of the phase transition properties of water. At 100 °C, liquid water possesses a specific enthalpy of approximately 419 kJ/kg, whereas steam at 100 °C has a significantly higher specific enthalpy of about 2,675 kJ/kg due to the latent heat of vaporization (approximately 2,256 kJ/kg). This difference in energy content makes steam significantly more efficient at delivering thermal energy compared to liquid water. Utilizing this characteristic allows for the effective and controlled ablation of target tissues while minimizing the required volume of water.

During use, once the saline exits lumen 104, it contacts an activated RF electrode 106 within the confined chamber 102, thereby undergoing rapid vaporization to create vapor 108. In some embodiments, electrode 106 comprises bipolar pairs of electrodes, that further include gaps or slots or perforations between the pairs for vapor to escape and/or for the fluid to flow. In some embodiments electrode 106 is open at either ends, thus allowing for fluid to flow. Due to confinement in chamber 102, vapor 108 reheats residual condensate and dry steam preferentially migrates radially outward. As water vapor exits chamber 102, dissolved salts remain behind, resulting in an increase in local salinity. This salinity increase influences electrical impedance at the electrode.

Porous membrane vapor filter 110 surrounds vaporization chamber 102 and comprises hydrophobic microporous material such as expanded polytetrafluoroethylene (ePTFE), high permeability polymeric matrices, or Tyvek/GORE-TEX. Porous membrane vapor filter 110 provides a selective outlet for the vapor 108 with a porosity that can range from 5% to 95%. The generated vapor 108 is delivered through layer 110, which encompasses RF electrode 106. Condensed liquid retained by porous membrane vapor filter 110 remains within chamber 102 and is reheated by electrode 106, promoting dry vapor output.

Any excess or residual liquid water from the vaporizing saline can impede thermal energy transfer by acting as a barrier between steam and tissue. Unlike steam, which efficiently transfers heat upon condensation, excess liquid water cools the surrounding tissue, thereby reducing the treatment's efficacy. Removing excess water from the ablation site is challenging and can compromise procedural efficiency. Embodiments of the present specification resolve these concerns by incorporating porous membrane vapor filter 110. Materials comprising porous membrane vapor filter 110 enable selective vapor passage while effectively repelling liquid water, ensuring that only high-energy, dry steam reaches the targeted tissue. The microporous structure of porous membrane vapor filter 110 ensures that liquid-phase water is retained and re-exposed to RF electrode 106 for reheating and vaporization. The present configuration enhances energy efficiency of the ablation catheter, ensures uniform vapor delivery, and determines overall safety of vapor ablation procedures.

The following are descriptions of some of the exemplary embodiments of the configurations comprising the porous membrane vapor filter, the at least one electrode, and the fluid delivery lumen.

### Membrane Exposed to External Environment

In some embodiments, the porous membrane vapor filter is positioned at the outer surface of the catheter shaft such that the membrane is exposed directly to the external environment surrounding the catheter. In this configuration, vapor generated within the annular volume passes through the porous membrane filter and is released directly from the outer surface of the catheter shaft into the surrounding treatment region. In such embodiments, the porous membrane filter functions as both a phase-separation element and a vapor outlet.

### Membrane Inside Shaft With Downstream Ports

In some embodiments, the porous membrane filter is positioned within the catheter shaft and is not directly exposed to the external environment. In these embodiments, vapor generated in the annular volume passes through the porous membrane filter into an internal vapor chamber within the shaft. The vapor then travels through one or more vapor outlet ports or passages formed in the catheter shaft before being released into the surrounding environment. This configuration allows the porous membrane filter to remain protected within the catheter structure while still providing vapor-phase separation prior to vapor delivery through the outlet ports.

### Annular Volume Between Membrane and Electrode

In some embodiments, the annular volume is defined between an inner surface of the porous membrane filter and an outer surface of the at least one electrode. In this configuration, the electrode is positioned radially inward of the porous membrane filter such that the vaporization region exists directly between the electrode surface and the membrane. Working fluid introduced into the annular volume contacts the electrode surface and is heated to generate vapor, which passes radially outward through the porous membrane filter.

### Annular Volume Between Membrane and Fluid Delivery Lumen

In some embodiments, the annular volume is defined between an inner surface of the porous membrane vapor filter and an outer surface of the fluid delivery lumen. In this configuration, the fluid delivery lumen extends through a central region of the catheter and the porous membrane filter is positioned radially outward from the lumen. The annular volume surrounding the fluid delivery lumen forms the vaporization region in which working fluid exits the fluid delivery lumen and is heated by the at least one electrode to generate vapor prior to passing through the porous membrane vapor filter.

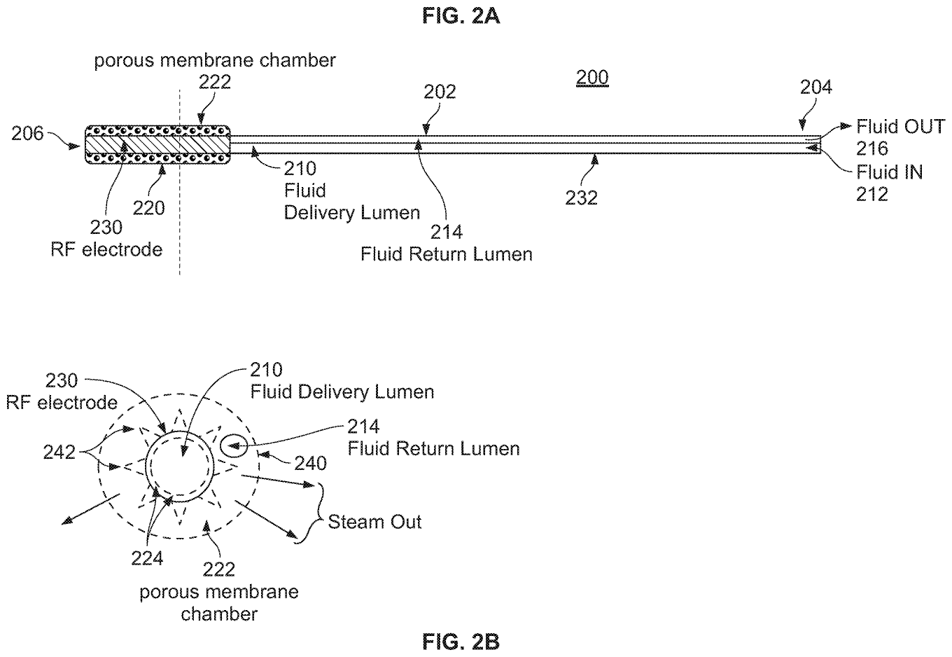

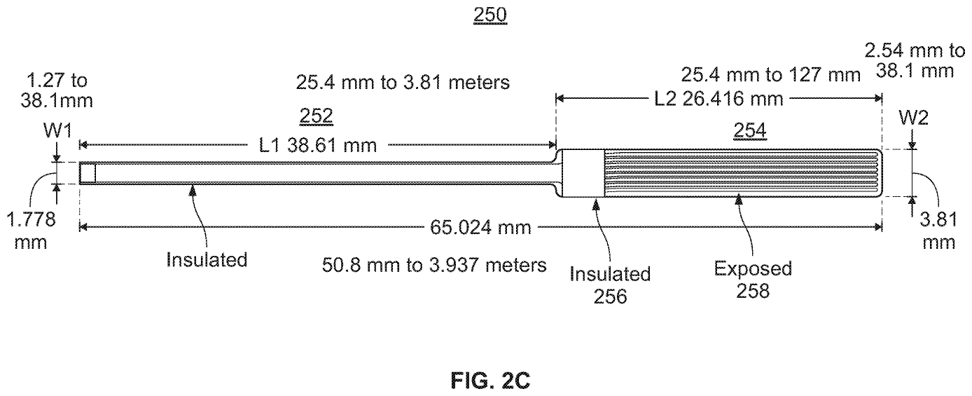

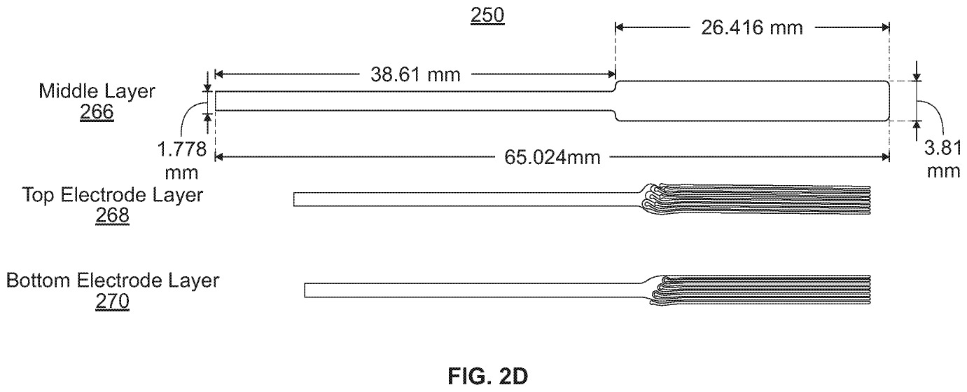

Referring now to FIGS. 2A and 2B, an example catheter 200 is shown for use in a catheter-based RF vapor generation system. FIG. 2A illustrates a longitudinal cross-section view of catheter 200, in accordance with some embodiments of the present specification. FIG. 2B illustrates a lateral cross-section view of a distal portion of catheter 200 comprising the RF vapor generation system in accordance with some embodiments of the present specification. Catheter 200 includes an elongate shaft 202 extending between a proximal region 204 and a distal region 206. Shaft 202 defines multiple lumens including: a fluid delivery lumen 210 and a fluid return lumen 214. Lumens 210 and 214 are parallel to each other. In some embodiments, lumen 210 has a first diameter D1 greater than a second diameter D2 of lumen 214. Working fluid enters through a proximal inlet 212 and travels toward a distal vaporization chamber 220. Within the distal region, catheter 200 includes: a plurality of delivery ports 240 for distributing working fluid, an RF electrode 230 configured to heat the fluid, electrical conductors 232 connecting the electrode to an RF generator, and a porous membrane vapor filter 240 forming a phase-separation boundary. The porous membrane vapor filter 240 surrounds the vaporization region and allows vapor to exit while retaining liquid. The space between electrode 230 and porous membrane vapor filter 240 forms a porous membrane chamber 222. In some embodiments, the chamber further includes structural supports 242 such as ribs, spacers, stand-offs, spikes, a perforated sleeve, a braid, a coil support, and/or a support cage. In some embodiments, the structural supports 242 further define and maintain a working-fluid layer adjacent RF electrode 230 and porous membrane vapor filter 240 to promote consistent electrical coupling and heat transfer from RF electrode 230 into the working fluid. In some embodiments, a thickness of the working-fluid layer between RF electrode 230 and porous membrane vapor filter 240 is selected relative to a distance or space 391 between individual traces 390 of the RF electrode (inter-trace distance or space 391). For example, the thickness may be from about one-half (½×) of the inter-trace distance 391 to about five times (5×) the inter-trace distance or space 391.

The at least one electrode 230 is not rigidly fixed between the fluid delivery lumen and the porous membrane vapor filter 240, but is positioned in a manner that allows limited movement or “floating” within the annular volume. As working fluid is introduced through the fluid delivery lumen and into the vaporization region, the fluid fills the space between the fluid delivery lumen, the electrode, and the porous membrane filter. The resulting fluid layer flows between these elements and tends to coaxially center electrode 230 relative to the fluid delivery lumen and the surrounding porous membrane vapor filter 240. This floating configuration allows electrode 230 to self-align within chamber 222 while maintaining a working-fluid layer between electrode 230 and adjacent structures, thereby promoting uniform electrical coupling and consistent vapor generation. In some embodiments, the one or more structural supports 242 can be used to limit excessive displacement of electrode 230 while still permitting electrode 230 to remain partially fluid-supported within the vaporization region.

Porous membrane chamber 222 is configured to receive the working fluid delivered via delivery lumen 210 and generate heated vapor and/or a vapor-rich two-phase mixture. Plurality of distal ports 240 forming a periphery of delivery lumen 210, distribute the working fluid into porous membrane chamber 222, thereby promoting wetting of an RF electrode 230 and improving repeatability of vapor generation. The plurality of ports 224 may comprise discrete apertures, slots, porous sections, or other flow-distribution features arranged circumferentially and/or axially.

RF electrode 230 is configured to receive RF energy from an external RF generator, and to heat the working fluid within porous membrane chamber 222 by ionic conduction and/or dielectric losses, thereby causing rapid heating and vaporization. In some embodiments, RF electrode 230 comprises a bipolar electrode arrangement. In some embodiments, RF electrode 230 is implemented as a patterned conductive element, a flex circuit, a wire or foil electrode, a plated or sputtered conductor, or another electrode configuration suitable for intraluminal use. Electrical conductors 232 extend proximally within shaft 202 to provide electrical coupling between RF electrode 230 and a proximal RF generator.

Porous membrane vapor filter 240 surrounds at least a portion of delivery lumen 210 and RF electrode 230 to define a vaporization chamber. In some embodiments, porous membrane vapor filter 240 comprises a hydrophobic vapor-permeable and liquid-restrictive membrane having structural properties such as pore size, void fraction, thickness, wettability, tortuosity, and effective open area selected to permit vapor passage while resisting liquid breakthrough, as described above with respect to FIG. 1. Porous membrane vapor filter 240 is configured to permit passage of vapor while restricting passage of bulk liquid. In some embodiments, porous membrane vapor filter 240 comprises a vapor-permeable and liquid-impermeable or liquid-restrictive membrane that preferentially allows vapor to pass outward (Steam Out) while substantially retaining liquid-phase working fluid and condensate within the porous membrane chamber 222. In some embodiments, the porous membrane vapor filter 240 comprises a hydrophobic porous material including, as non-limiting examples, a nonwoven polymer (such as, flash-spun HDPE), a microporous polymer, porous PTFE, or ePTFE. In some embodiments, the porous membrane vapor filter 240 includes pores sized to retain condensate while permitting vapor flow, for example in a pore-size range from about 0.1 μm to about 1.0 μm, although other pore sizes and constructions are contemplated depending on target vapor quality, pressure, and flow conditions.

In some embodiments, retaining liquid within porous membrane chamber 222 provides a condensation-management mechanism in which condensate remains in thermal communication with RF electrode 230 for reheating and revaporization. In some embodiments, this configuration supports delivery of higher vapor quality by reducing or preventing expulsion of bulk liquid from the chamber.

In some embodiments, return lumen 214 is in fluid communication with porous membrane chamber 222 and is configured to exhaust liquid from the porous membrane chamber 222. In an exemplary situation, as water is vaporized and exits through porous membrane vapor filter 240, solute concentration of retained liquid may increase. Return lumen 214 may be used to remove concentrated working fluid and/or retained liquid from the porous membrane chamber 222, including during a flush cycle. In some embodiments, a flush cycle includes introducing fresh working fluid via delivery lumen 210 while exhausting concentrated working fluid via return lumen 214. In some embodiments the rate of flush is configured to be between 0.1 ml/min and 10 ml/min. In some embodiments, exhausted fluid is directed to waste through proximal outlet 216. In some embodiments, exhausted fluid is directed to a reservoir for recirculation, optionally including filtration, degassing, dilution, conductivity control, and/or temperature conditioning performed external to catheter 200.

Referring to FIG. 2B, in some embodiments, delivery lumen 210 is arranged centrally within shaft 202, while return lumen 214 is arranged offset from a central axis. In some embodiments, RF electrode 230 is positioned around delivery lumen 210 within porous membrane chamber 222 to promote rapid heating of fluid delivered through ports 224. In some embodiments, porous membrane vapor filter 240 circumscribes the distal region to define an annular vapor outlet region through which vapor exits (Steam Out).

In operation, RF electrode 230 and working fluid within porous membrane chamber 222 present an electrode-fluid load impedance measurable by the external RF energy control system. In some embodiments, impedance is monitored as a proxy for changing salinity and/or conductivity of the working fluid over time and across repeated treatments. The RF energy control system may adjust RF delivery and/or fluid flow based on measured impedance changes and may initiate a flush cycle when an impedance-derived threshold condition is met (for example corresponding to a threshold salinity level) and may stop the flush cycle when impedance returns toward a baseline condition. In embodiments, a baseline impedance level ranges from 5 to 25 ohms. Furthermore, the threshold impedance value is configured at a level≥50% of the baseline impedance, in one embodiment. In another embodiment, the threshold value is configured to be ≥25% of the baseline impedance.

Although FIGS. 2A and 2B illustrate one non-limiting configuration, catheter 200 may be implemented with any suitable arrangement of lumens, ports, electrodes, and porous membrane vapor filter structures. For example, porous membrane chamber 222 may be positioned at the distal tip or at a distal shaft region in fluid communication with a distal outlet; delivery ports 224 may be configured to distribute flow circumferentially and/or axially; and return lumen 214 may be configured to remove liquid continuously or intermittently. Any feature described herein may be used alone or in combination with other embodiments unless otherwise indicated.

In certain embodiments, deionized water and/or distilled water may be flushed through return lumen 214 to dilute concentrated working fluid (for example, concentrated saline) and adjust salinity toward a baseline level or a selected therapeutic range. In embodiments, a duration of the flush is configured based on the flow rate of the saline and the time required to achieve the salinity within a normal range.

FIG. 2C illustrates an exemplary design of an electrode configuration 250 used for heating an ablation fluid for thermal ablation in accordance with some embodiments of the present specification. The design comprises an elongated rectangular body with a first portion 252 having a first length L1 ranging from 25.4 mm to 3.81 meters, and is 38.61 mm in an embodiment, and having a first width W1 ranging from 1.27 to 38.1 mm, and is 1.778 mm in an embodiment. First portion is continually and linearly connected to a second portion 254 having a second length L2 ranging from 25.4 mm to 127 mm, and is 26.416 mm in an embodiment, and having a second width W2 ranging from 2.54 mm to 38.1 mm, which is 3.81 mm in an embodiment. Therefore, a total length of the electrode configuration ranges from 50.8 mm to 3.937 meters, and is 65.024 mm in an embodiment. Outer surface of first portion 252 is insulated, whereas a portion 256 of outer surface of second portion 254, which is proximal to first portion 242 is insulated, while the surface of the remaining portion 258 is exposed.

FIG. 2D separately illustrates the different layers used to form the electrode configuration 250 of FIG. 2C, in accordance with some embodiments of the present specification. Three layers form the configuration, comprising a middle layer 266 which is between a top electrode layer 268 and a bottom electrode layer 270. Base material of each electrode layer is 34.79 μm cu/25 μm polyimide/34.79 μm cu. A cover layer covering both sides of the electrode layers is 12 μm polyimide, which extends up to 25 μm of both sides. Parts of the electrode layers are copper plated. Finished thickness of each layer is approximately 34.79 μm cu.

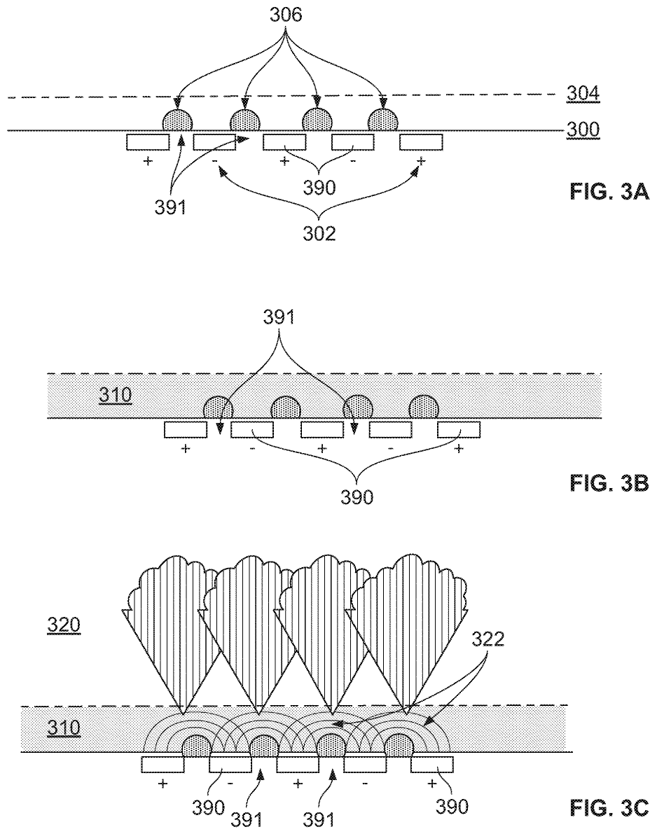

Referring to FIGS. 3A-3C, an example distal vapor generation configuration is illustrated in which a flexible RF heater strip 300 is positioned proximate a porous membrane vapor filter 304 to define a vapor generation chamber therebetween.

Referring to FIG. 3A, flexible RF heater strip 300 includes a plurality of RF electrodes 302 arranged along the strip. In some embodiments, RF electrodes 302 comprise a bipolar electrode arrangement (alternating polarity electrode segments) configured to couple RF energy into an ionic working fluid. Porous membrane vapor filter 304 is positioned above (or otherwise parallel and proximal to) flexible RF heater strip 300 and is configured to permit passage of vapor while restricting passage of bulk liquid.

In some embodiments, one or more structural support members 306 (spacers, ribs, stand-offs, protrusions, or other types of support features) are provided to define and maintain a gap between RF electrodes 302 and porous membrane vapor filter 304. In some embodiments, the gap is selected to promote electrical coupling and heat transfer from the RF electrodes 302 into the working fluid while maintaining vapor permeability of the porous membrane vapor filter 304 and limiting collapse of the chamber. In one non-limiting, but preferred, embodiment, a thickness of the gap (and thus a thickness of a working-fluid layer) is about two times (2×) the inter-trace distance or space 391 between adjacent traces 390 of the RF electrode 302, although other thicknesses and relationships as described previously are contemplated. In other embodiments, the thickness of the gap is about 0.2 to 1.9 times (2×) the inter-trace distance or space 391. In other embodiments, the thickness of the gap is about 2 to 8 times (and every whole number increment therein) the inter-trace distance or space 391. In other embodiments, the thickness of the gap is about 0.2 to 10 times (and every whole number increment therein) the inter-trace distance or space 391.

In some embodiments, RF heater strip 300 is implemented as a flexible printed circuit (“flex strip”) comprising a bipolar electrode configuration for RF electrodes 302. The bipolar electrode configuration is formed by first and second conductive traces 390 on a polyimide substrate. In some embodiments, the conductive traces 390 have a width of about 0.4 mm and an inter-trace distance, or space, 391 of about 0.2 mm, and the polyimide substrate has a thickness in a range from about 25 μm to about 50 μm. In other embodiments, the conductive traces 390 have a width in a range of 0.1 mm to 0.9 mm and an inter-trace distance, or space, 391 of about 0.02 mm to 2 mm. In some embodiments, the bipolar electrode configuration is flexible and can tolerate repeated bending, including to bend radii on the order of about 2 mm, to facilitate navigation through tortuous anatomy.

In some embodiments, electrodes 302 include one or more surface treatments to improve heating uniformity and reduce edge-localized current density, including rounded electrode edges and/or increased effective surface area (such as, nanoporous surface structures) and/or conductive coatings configured to reduce electrode-fluid interfacial impedance.

Referring to FIG. 3B, a working fluid 310, such as saline, is introduced into the chamber between the flexible RF heater strip 300 and the porous membrane vapor filter 304. In some embodiments, working fluid 310 flows into the chamber via a delivery lumen (not shown in FIGS. 3A-3C) and forms an “optimal thickness” working-fluid layer supported by structural support members 306. In some embodiments, maintaining a selected working-fluid layer thickness promotes consistent electrical coupling between electrodes 302 and working fluid 310 and supports repeatable heating performance and vapor generation.

In one embodiment, the structural support members is an amount of material extending perpendicularly from a surface of the electrode structure. That material may adopt any shape provided it does not materially block a surface of one or more of the traces 390 of the electrode and provided it extends far enough to form the desired thickness of the working fluid layer. Preferably, one or more of the structural support members are positioned in the interelectrode

Referring to FIG. 3C, RF energy 322 is delivered to electrodes 302 such that energy couples through working fluid 310 and heats the working fluid within the chamber. In some embodiments, the selected working-fluid layer thickness enhances coupling and reduces time-to-vaporization relative to thicker or thinner layers. As working fluid 310 is heated, vapor 320 is generated within the chamber and exits through porous membrane vapor filter 304. In some embodiments, the configuration illustrated in FIG. 3C supports higher vapor production and/or more uniform vapor generation along the electrode array by maintaining a consistent fluid layer thickness and by distributing heating across multiple electrode segments.

### Condensation Recycling

In embodiments of the present specification as described above, porous membrane vapor filter 110/240/304 (see FIGS. 1, 2A, 2B, 3A-3C) retains liquid condensate within chamber 102/222, so that condensed droplets remain in contact with RF electrode 106/230/302. RF electrode 106/230/302 enables continuous reheating of condensed liquid and its conversion back into vapor. Therefore, embodiments of the present specification enable: reheating, re-vaporization, increased dryness of output vapor, and improved thermal efficiency. The coaxial chamber thus operates as a localized vapor recirculation environment.

### Gas Infusion Embodiment

Additionally, embodiments of the present specification introduce a gas such as carbon dioxide (CO₂) into the saline delivery lumen. In some embodiments, CO₂ is introduced at a controlled flow rate along with saline into the vaporization chamber. The flow rate can range from 0.5-10 L/min (1-50 psi). The RF vaporization process heats the CO₂ along with the saline. The gas may either mix with saline prior to vaporization, or may enter the chamber in parallel with saline. The heated CO₂ is released along with water vapor through the porous membrane vapor filter. The advantage of delivering heated CO₂ is its ability to retain thermal energy over a longer distance (enhanced thermal propagation) compared to water vapor, ensuring deeper tissue heating and improved uniformity in energy transfer. In some embodiments, infusion of gas is regulated by a flow controller and may operate continuously or intermittently.

A gas (such as CO₂) is co-delivered with the working fluid into the vaporization region. The gas may serve as a carrier/transport medium, may shape pressure and flow dynamics, may displace blood or other fluids, may improve visualization under imaging, and/or may reduce condensation within portions of the delivery pathway by altering partial pressures and convective transport. The gas may be heated in the vaporization region and co-exhausted with vapor through the porous membrane vapor filter. These embodiments are optional and may be used to improve delivery consistency and procedural workflow.

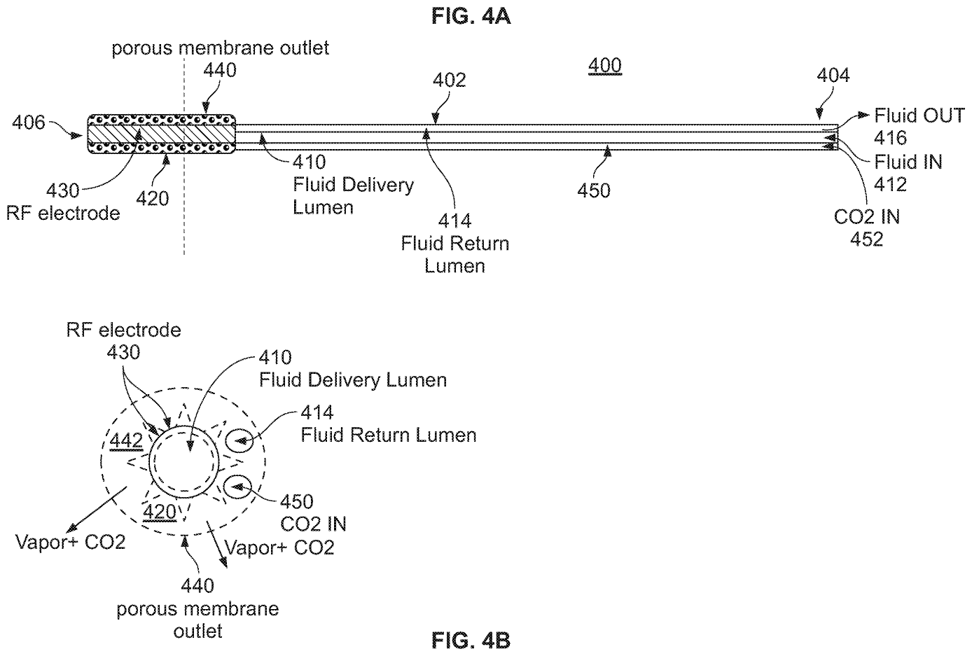

Referring to FIGS. 4A and 4B, an example catheter 400 is shown that is the same as, or generally analogous to, catheter 200 described with respect to FIGS. 2A and 2B, except that catheter 400 further includes a lumen 450 configured to deliver a gas (such as CO₂) to the distal vapor generation chamber. In some embodiments, the gas may comprise CO₂, N₂, O₂, or another gas, and the selection may be based on delivery characteristics, imaging considerations, condensation management, and/or desired thermal transport behavior.

FIG. 4A illustrates a longitudinal cross-section view of catheter 400, similar to FIG. 2A, in accordance with some embodiments of the present specification. FIG. 4B illustrates a lateral cross-section view of a distal section of catheter 400, similar to FIG. 2B, in accordance with some embodiments of the present specification. Referring to FIG. 4A, catheter 400 includes an elongate shaft 402 extending between a proximal region 404 and a distal region 406. Shaft 402 defines a fluid delivery lumen 410 configured to convey a working fluid from a proximal inlet (Fluid IN) 412 to a distal vapor generation chamber 420. Shaft 402 further defines a fluid return lumen 414 configured to convey return fluid from the distal region 406 toward a proximal outlet (Fluid OUT) 416. In some embodiments, an RF electrode 430 is positioned at or near distal vapor generation chamber 420 such that RF energy heats the working fluid to generate heated vapor and/or a vapor-rich two-phase mixture within chamber 420. Chamber 420 is at least partially bounded by a porous membrane vapor filter 440 configured to permit passage of vapor while restricting passage of bulk liquid, thereby promoting delivery of vapor and improving vapor quality as described above with respect to FIGS. 2A and 2B.

FIG. 4A further illustrates that catheter 400 includes a gas lumen 450 configured to convey a gas from a proximal gas inlet (CO₂ IN) 452 to the distal vapor generation chamber 420. In some embodiments, gas lumen 450 terminates at one or more distal gas ports positioned to introduce gas directly into the vapor generation chamber 420. In some embodiments, gas introduced via gas lumen 450 mixes with vapor generated in chamber 420 to form a heated gas-vapor mixture that is expelled outward through the porous membrane vapor filter 440. In some embodiments, the expelled mixture may comprise vapor+gas (for example, Vapor+CO₂) and exits circumferentially through the porous membrane vapor filter 440.

Referring to FIG. 4B, a cross-sectional configuration is illustrated in which fluid delivery lumen 410 is arranged to deliver working fluid into vapor generation chamber 420, fluid return lumen 414 is arranged to remove liquid from chamber 420 (including concentrated working fluid and/or condensate), and gas lumen 450 is arranged to deliver gas into chamber 420. In some embodiments, fluid delivery lumen 410 is positioned centrally and fluid return lumen 414 and gas lumen 450 are positioned radially offset, although other lumen arrangements are contemplated. In some embodiments, RF electrode 430 is positioned proximate to fluid delivery lumen 410 such that delivered working fluid rapidly heats within chamber 420.

In some embodiments, adding gas lumen 450 and introducing a gas into chamber 420 modifies delivery characteristics relative to vapor alone. In some embodiments, vapor generated in chamber 420 may partially condense upon cooling after exiting the porous membrane vapor filter 440, and such condensation can reduce the distance over which latent heat is transported. In some embodiments, the introduced gas does not condense under use conditions and can therefore act as a carrier to convectively transport thermal energy and/or the vapor-gas mixture farther from the outlet than vapor alone, while also shaping flow and pressure dynamics and reducing condensation by altering partial pressure and residence time. In some embodiments, the gas additionally displaces blood locally and/or improves imaging visibility depending on the procedure and imaging modality.

In some embodiments, catheter 400 optionally utilizes return lumen 414 for intermittent or continuous removal of liquid from chamber 420, including to manage solute concentration (such as, salinity increase) associated with vapor generation. In some embodiments, gas lumen 450 may be operated continuously or intermittently and may be coordinated with RF delivery and/or flush cycles, including to maintain stable vapor generation efficiency and consistent delivery of the heated gas-vapor mixture through porous membrane vapor filter 440.

### Electrical Control System

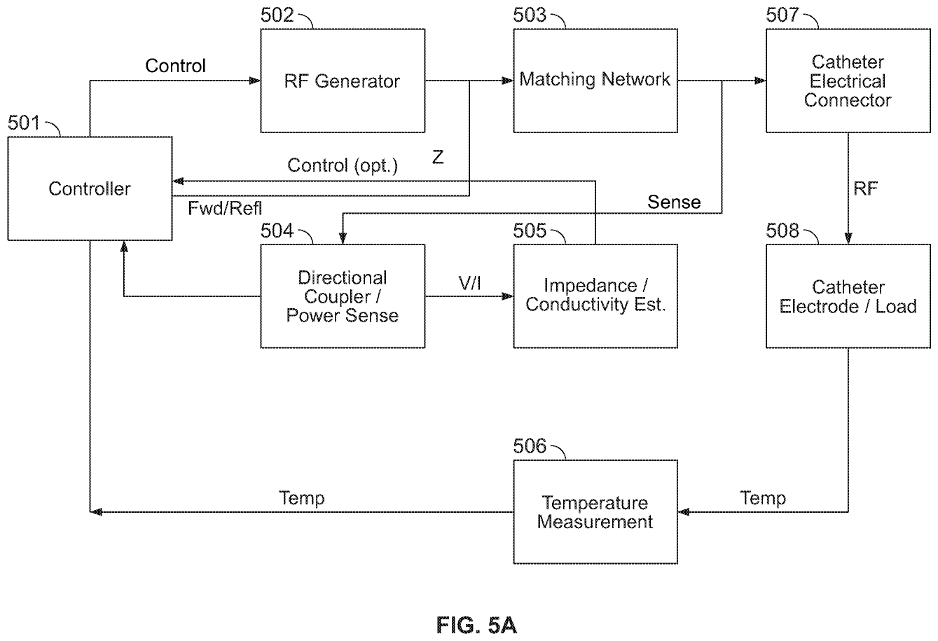

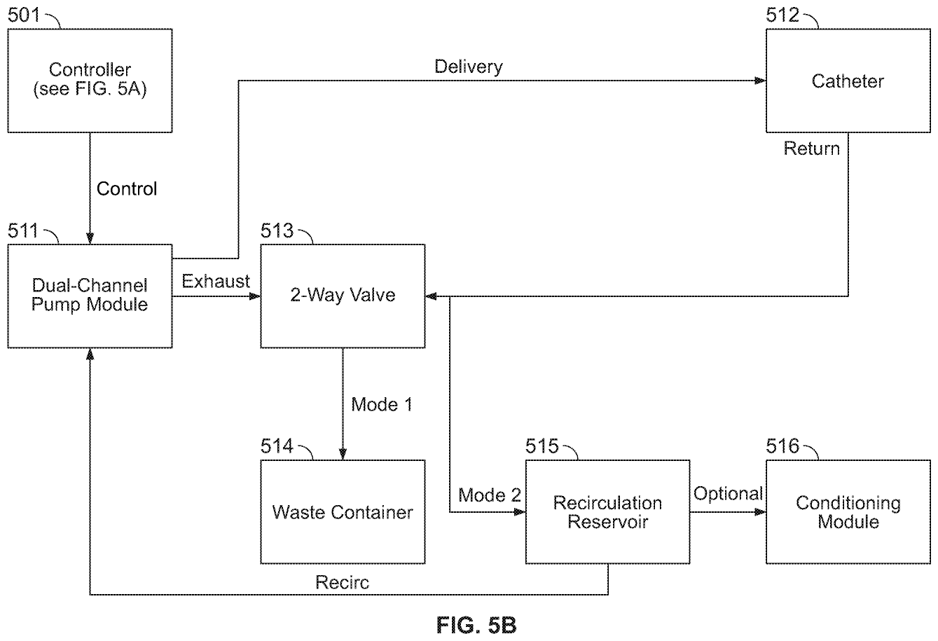

FIG. 5A illustrates an electrical and control architecture for a catheter-based RF vapor generation system configured to improve vapor quality, heating efficiency, and controllability using electrical-parameter sensing and closed-loop control, in accordance with some embodiments of the present specification. In some embodiments, the system adjusts one or more RF delivery parameters based on measured electrical parameters to maintain heating performance and vapor generation efficiency. For example, the system may adjust frequency, output power, and/or duty cycle responsive to measured impedance, reflected power, forward power, phase, or combinations thereof, including to compensate for changes in fluid conductivity during heating. The system includes: a controller 501, an RF generator 502, a matching network 503, a directional coupler/power sensing module 504, an impedance estimator 505, a temperature measurement module 506, a catheter electrical connector 507, and an electrode-fluid load 508.

Controller 501 executes workflow sequencing and closed-loop control and may also implement user interface functions, data logging, and safety interlocks. Controller 501 outputs control commands to RF Generator 502 to regulate one or more RF delivery parameters, including output power, duty cycle, and operating frequency.

In some embodiments, RF Generator 502 operates at a selected frequency suitable for heating ionic working fluids, including frequencies selected from a broad range (for example about 10 kHz to about 100 MHz, including subranges such as about 100 kHz to about 30 MHz and about 1 MHz to about 27 MHz), with about 13.56 MHz as one non-limiting example. In some embodiments, RF Generator 502 delivers RF energy in a continuous mode or in a pulsed mode. In pulsed embodiments, pulse widths may be selected from a broad range (such as, for example, approximately 1 microsecond to approximately 10 milliseconds), including a non-limiting example range of approximately 10 microseconds to approximately 100 microseconds, and duty cycle may be limited (such as, below approximately 20%, below approximately 10%, or below approximately 5%) to achieve rapid heating while reducing localized overheating.

RF output from RF Generator 502 is provided to matching network 503. Matching network 503 is configured to match a source impedance (nominally approximately 25 ohms in some embodiments) to the catheter electrode-fluid load to improve power transfer and reduce reflected power. Matching network 503 may be fixed, switchable, or tunable. In tunable embodiments, controller 501 may command a tuning actuator associated with matching network 503 (such as, switched capacitor/inductor banks, varactors, motor-driven capacitors, digitally controlled matching elements, or combinations thereof) to adjust matching based on measured electrical parameters. In some embodiments, the system tunes to reduce reflected power and/or to maintain a voltage standing wave ratio (VSWR) below a threshold, such as below about 2:1, below about 1.5:1, or in some preferred embodiments below about 1.2:1.

RF energy is delivered from matching network 503 to catheter electrical connector 507, which provides the electrical interface between the system and the catheter. Catheter electrical connector 507 may include one or more conductors for RF power delivery and may optionally include identification and/or keying features (such as, resistor coding, memory device, pin pattern, mechanical keying, RFID) used to enable/disable therapy modes, apply limits, or configure parameter ranges. The RF output energizes catheter electrode/load 508, which represents the effective electrode-fluid electrical load present during vapor generation. Catheter electrode/load 508 may include one or more electrodes positioned within or adjacent a vaporization region and in electrical contact with an ionic working fluid. The magnitude and phase of the electrode-fluid load impedance may vary during operation due to changes in working fluid conductivity, temperature, phase state, and solute concentration.

Directional coupler/power sense 504 is coupled to the RF path (such as, via a directional coupler, current/voltage sensing network, or other sensing tap) and measures one or more electrical parameters indicative of load behavior and match quality. Non-limiting examples include forward power, reflected power, phase, and/or voltage and current. Outputs from directional coupler/power sense 504 are provided to impedance/conductivity estimator 505. Impedance/conductivity estimator 505 computes impedance magnitude and/or phase and may also generate derived proxies such as conductivity estimates, salinity proxies, and/or indicators of vaporization state. The derived proxies may be generated using calibration tables, empirical correlations, model-based estimation, and/or real-time measurement of V/I and phase.

Controller 501 receives one or more feedback signals from directional coupler/power sense 504 and/or impedance/conductivity estimator 505 and adjusts RF delivery accordingly. In some embodiments, controller 501 adjusts RF power, duty cycle, frequency, and/or matching to maintain vapor generation efficiency and stable output in the presence of conductivity drift. In some embodiments, controller 501 maintains operation within a preferred impedance region associated with increased power available at the electrode and reduced time-to-target temperature. Broadly, operable electrode-fluid impedance may span a wide range (for example about 0.5 ohms to about 500 ohms, including about 1 ohm to about 200 ohms). Preferred operating windows may be centered near about 10 ohms, including non-limiting preferred windows of about 5 -15 ohms, 6-30 ohms, 10-25 ohms, and/or about 5-30 ohms, while maintaining operability outside those preferred windows. In some embodiments, controller 501 uses impedance and/or reflected power metrics to trigger control actions such as reducing duty cycle, adjusting frequency, initiating a matching retune, and/or modifying fluid flow conditions to maintain heating performance within safety constraints.

Temperature measurement 506 receives one or more temperature sensor signals associated with the catheter and provides a temperature signal to controller 501. Non-limiting examples include thermocouples, RTDs, thermistors, and fiber optic temperature sensors. Temperature sensing may be positioned at any suitable location, including within a working-fluid region, near the electrode, within the catheter shaft, or at a distal treatment region, depending on the embodiment. Controller 501 uses the temperature signal for closed-loop thermal regulation, for example to control time-to-target temperature, to maintain a selected temperature band, and to enforce safety shutdown for over-temperature or sensor faults.

Together, the blocks of FIG. 5A describe an integrated system in which RF energy delivery, impedance-aware estimation, matching optimization, and temperature feedback operate under controller supervision to provide rapid and controlled heating of an ionic working fluid for vapor generation while maintaining safety and repeatability.

### Fluidics Control Architecture

FIG. 5B illustrates a fluidics architecture for delivering working fluid to a catheter vaporization region and selectively directing return fluid to waste or to a recirculation loop, under the control of controller 501 (see FIG. 5A).

A Dual-Channel Pump Module 511 provides controlled fluid movement for two primary functions: (i) delivery of fresh working fluid to the catheter and (ii) removal and management of return fluid, including flush operation. In the illustrated embodiment, the pumping and valving functions are integrated into dual-channel pump module 511. In other embodiments, the same functions are implemented using two separate pumps and associated valves (such as, a delivery pump and a return/flush pump) or using alternative pump types (such as, syringe pumps, piston pumps, peristaltic pumps, diaphragm pumps), without limitation.

A Catheter 512 includes at least a delivery lumen and an optional return lumen in fluid communication with a distal vaporization region. Dual-Channel Pump Module 511 delivers working fluid through the delivery lumen to support vapor generation at the distal vaporization region. Return fluid from the catheter is received through the return lumen. Return fluid may include concentrated working fluid (such as, increased solute concentration after vapor generation), condensate, and/or mixed-phase fluid depending on operating conditions.

In some embodiments, the system initiates a flush cycle by delivering fresh working fluid through the delivery lumen while exhausting concentrated working fluid through the return lumen. Flush initiation may be triggered based on measured electrical parameters (such as, impedance magnitude/phase, reflected power, conductivity proxy, salinity proxy) indicating drift from a desired operating condition, based on time, based on delivered vapor volume, based on heating-rate degradation, and/or based on user selection. Flush termination may occur when electrical parameters return to a baseline condition, when a target exchanged volume has been achieved, or when a maximum flush duration is reached.

Return flow is routed through a 2-Way Valve 513 (or another flow-directing element such as a manifold, shuttle valve, solenoid valve, pinch valve arrangement, or rotary valve). 2-Way Valve 513 selects between at least two return paths:

Mode 1 (exhaust-to-waste): return fluid is directed to Waste Container 514. This embodiment removes concentrated working fluid from the system, thereby limiting solute accumulation and stabilizing vapor generation efficiency.

Mode 2 (recirculation): return fluid is directed to Recirculation Reservoir 515 for reuse. In recirculation embodiments, Recirculation Reservoir 515 may store return fluid and supply fluid back to Dual-Channel Pump Module 511 via a recirculation loop. In some embodiments, Controller 501 selects between waste and recirculation modes based on one or more criteria such as impedance-based salinity estimation, conductivity proxy, reflected power, elapsed time, delivered vapor volume, procedure phase, or user input.

Conditioning Module 516 is optionally coupled to the recirculation path to modify one or more properties of the working fluid prior to reuse. Non-limiting examples of conditioning include filtration (such as, particulate removal), degassing (such as, dissolved gas reduction), dilution with fresh working fluid, ion exchange or conductivity control, temperature conditioning (heating/cooling), and combinations thereof. Conditioning may be continuous, intermittent, or invoked during flush cycles. In some embodiments, the system uses conditioning to maintain the working fluid within a target conductivity range that supports preferred electrode-fluid impedance behavior and rapid heating.

Controller 501 commands Dual-Channel Pump Module 511 and, where present, 2-Way Valve 513 and Conditioning Module 516 to implement delivery, return, flush, and mode selection behaviors. In some embodiments, the controller selects Mode 1 or Mode 2 dynamically based on real-time electrical and thermal feedback (FIG. 5A), thereby maintaining vapor quality and energy efficiency while providing flexibility between single-pass waste disposal and closed-loop recirculation operation.

In some embodiments, instead of initiating a flush cycle that exhausts concentrated working fluid through return lumen 214/414, the system includes a source-selection valve (such as, a switch valve, manifold valve, or multi-port valve) configured to selectively couple the delivery lumen to a first working fluid source and to a second working fluid source. In one non-limiting embodiment, the first working fluid comprises saline (or another ionic fluid) and the second working fluid comprises deionized water and/or distilled water. In operation, the system may deliver the first working fluid during energy delivery and, upon detection of an electrical-parameter threshold condition (such as, impedance magnitude/phase drift or a salinity proxy exceeding a threshold such as about 5%), automatically switch the source-selection valve to deliver the second working fluid through the delivery lumen. Delivering the second working fluid dilutes the working fluid within the vaporization region and within the delivery/return flow path, thereby reducing conductivity and adjusting the electrode-fluid impedance toward a baseline or selected operating range. In some embodiments, after the impedance returns toward a baseline value or a target operating window, the system switches the source-selection valve back to the first working fluid source and resumes energy delivery. In some embodiments, the switch valve is commanded based on measured impedance, reflected power, a conductivity proxy, elapsed time, delivered vapor volume, heating-rate degradation, user selection, or combinations thereof. The baseline impedance value can be between 5 and 25 ohms in some embodiments, when the saline concentration is approximately 0.45%-0.9%. The baseline impedance value can depend on saline concentration levels and/or the electrode geometry, so that for one level of salinity, different electrode shapes will indicate different impedance. Therefore, in different electrodes, the ideal zone for heating and vaporization is different.

### Exemplary Process for Impedance Compensation

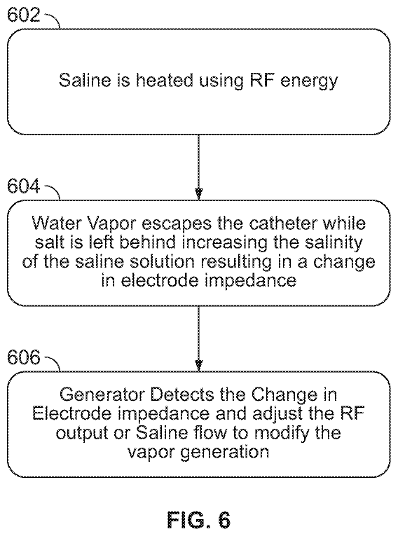

FIG. 6 is a flow chart illustrating an exemplary method to address changing electrode impedance due to the changing salinity in proximity to an RF electrode, in accordance with some embodiments of the present specification. At step 602, saline is heated using RF energy. Saline delivered through lumen 104 (see FIG. 1) exits via ports 112 and contacts electrode 106 within chamber 102. RF energy heats saline, producing vapor 108. As vapor 108 exits through membrane 110, at step 604, the water content decreases resulting in an increase in salt concentration. This further results in a change in electrode impedance. At step 606, the RF generator detects the change in impedance at RF electrode 106. The RF generator measures impedance changes in real time and provides the measured values to the controller for adaptive adjustment of RF delivery parameters. In response to detected impedance variation, the system adjusts at least one of: RF power, RF frequency, RF duty cycle, volume of the working fluid flow and/or working fluid or saline flow rate. This closed-loop control maintains efficient vapor generation despite changing salinity.

### Exemplary Flush Cycle Management Method

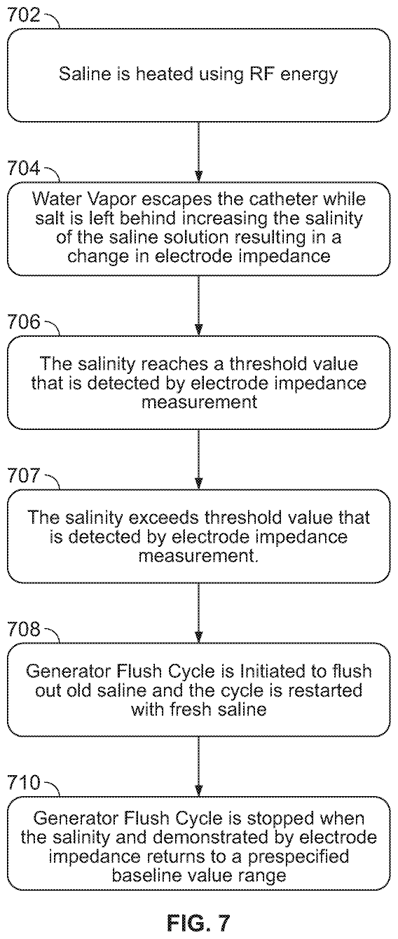

FIG. 7 is a flow chart illustrating an exemplary method to manage salinity when a threshold value of salinity is reached, in accordance with some embodiments of the present specification. At step 704, water vapor escapes the catheter while salt is left behind, which results in an increase in the salinity of the saline solution, which further results in a change in electrode impedance. Saline continues to vaporize within chamber 102, increasing salt concentration. At step 706, impedance measurements indicate salinity level. When impedance corresponds to a predetermined threshold concentration (for example exceeding approximately 5%, from an initial concentration of approximately 0.45%-0.9%), corrective action is evaluated. At step 707, as detected by the electrode impedance measurement, it is determined if the impedance exceeds a predetermined/programmed threshold value. At step 708, if the threshold is exceeded, a generator flush cycle is initiated to displace concentrated saline. Further, fresh saline is introduced through lumen 104. In some embodiments, RF output is reduced or paused during the flush. At step 710, The flush cycle terminates once impedance returns to a baseline corresponding to desired saline concentration. In some embodiments the desired saline concentration is approximately 0.45 to 0.9%. The vapor generation cycle then resumes.

### Energy Delivery Control

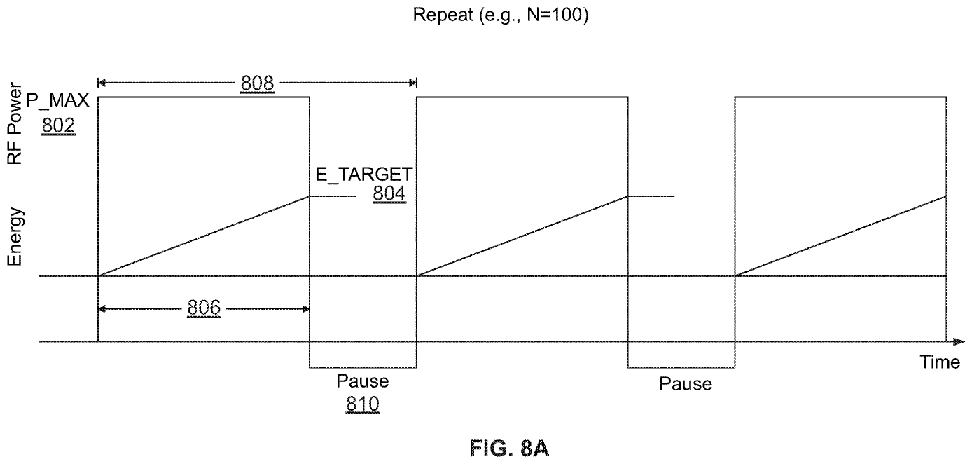

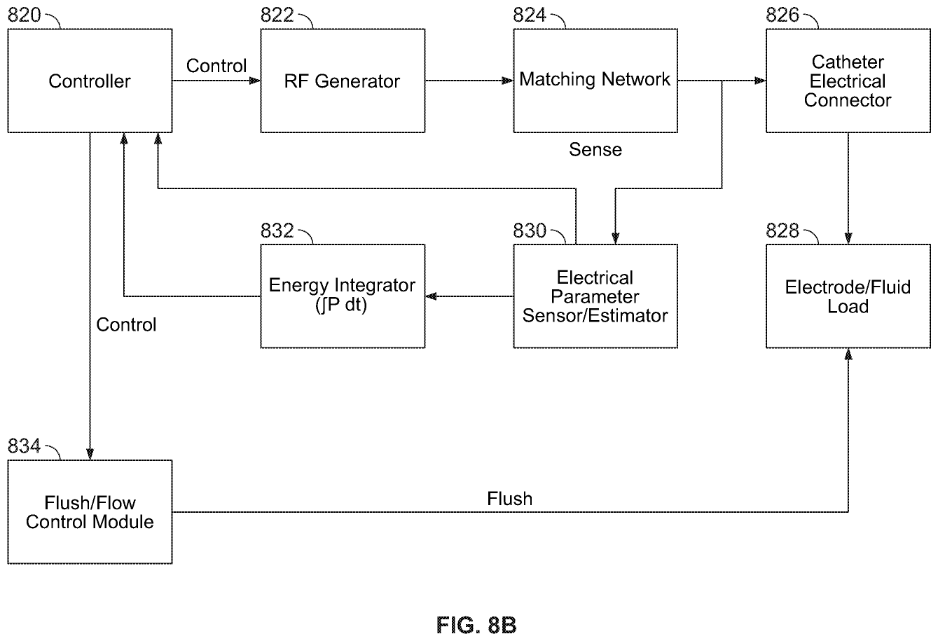

Referring to FIG. 8A, a timing relationship is shown between RF power delivered to an electrode/fluid load and accumulated delivered energy for a treatment cycle. In the illustrated embodiment, RF power is commanded to a high level, represented as P_MAX 802, for a delivery interval sufficient to reach a target delivered energy E_TARGET 804. In an embodiment, E-TARGET 804 is approximately 500 joules. Once E_TARGET 804 is reached, RF delivery is terminated such that accumulated delivered energy remains substantially constant thereafter. In some embodiments, a delivery interval 806 is selected to be short. In one embodiment, delivery interval 806 is on the order of approximately 5 seconds to deliver E_TARGET 804 in the shortest time subject to system and safety limits.

FIG. 8A further illustrates that a treatment cycle 808 may be repeated a plurality of times. In some embodiments, treatment cycle 808 is repeated N times, where N≈100. In some embodiments, an intervening pause 810 occurs between cycles, for example to reposition a catheter, with the pause being on the order of about 1-10 seconds and in some embodiments up to about 1 minute depending on the application. In some embodiments, repeated cycles increase salinity of a working fluid and thereby change electrode/fluid impedance, which can affect power transfer and time-to-delivery. In some embodiments, the system compensates for impedance drift such that E_TARGET 804 is still delivered per cycle and delivered in as short a period of time as practicable. In some embodiments, a flush or dilution operation is performed only when a threshold condition is met (for example corresponding to an inferred salinity threshold such as about 5%), as described with respect to FIG. 8B.

Referring to FIG. 8B, an example control architecture is shown for delivering a target electrical energy to an electrode/fluid load while adapting to changes in electrode/fluid impedance that may occur during vapor generation and across repeated cycles. Some of the elements illustrated in FIG. 8B overlap with the elements illustrated in FIG. 5A. It should be understood that these elements are common to the embodiments described in the present specification. Controller 820 generates control commands for RF Generator 822. RF Generator 822 delivers RF energy through Matching Network 824 and Catheter Electrical Connector 826 to the Electrode/Fluid Load 828. In some embodiments, the RF waveform comprises a standard RF electrosurgical waveform. In some embodiments, energy transfer during use proceeds from an RF electrode into an ionic working fluid (saline, for example), from the heated fluid into generated vapor, and from the vapor to tissue.

Electrical Parameter Sensor/Estimator 830 determines one or more electrical parameters associated with the Electrode/Fluid Load 828. In some embodiments, the Electrical Parameter Sensor/Estimator 830 measures voltage and current and estimates impedance and/or a proxy indicative of conductivity or salinity. The estimated electrical parameter is provided to Controller 820 (“Z”), so that Controller 802 adjusts RF delivery to accommodate impedance drift, including by adjusting delivered power and/or duty such that the target energy is delivered in the shortest time practicable subject to safety constraints.

Energy Integrator 832 determines delivered energy at the Electrode/Fluid Load 828 by integrating delivered power (P) over time (∫P dt). In the illustrated embodiment, delivered power (P) is provided to Energy Integrator 832, and Energy Integrator 832 provides an accumulated energy value (E) to Controller 820. In some embodiments, Controller 820 commands RF delivery at or near P_MAX 802 until the accumulated energy reaches E_TARGET 804, and terminates RF delivery once E_TARGET 804 is reached, thereby implementing a “full-on then off” energy-delivery strategy.

Flush/Flow Control Module 834 is controlled by Controller 820 and is configured to modify working fluid conditions that influence the Electrode/Fluid Load 828, including conductivity and impedance. In some embodiments, flushing is not performed every cycle; instead, flushing is initiated only when a threshold condition is met. In one embodiment, flushing is initiated when impedance behavior indicates an inferred salinity threshold such as about 5%. Further, the flushing is terminated when the measured or estimated electrical parameter returns toward a baseline condition. In other embodiments, Controller 820 continues to adapt RF delivery to accommodate increasing salinity or impedance drift without flushing, while still delivering E_TARGET 804 per cycle.

### Impedance and Vaporization Efficiency

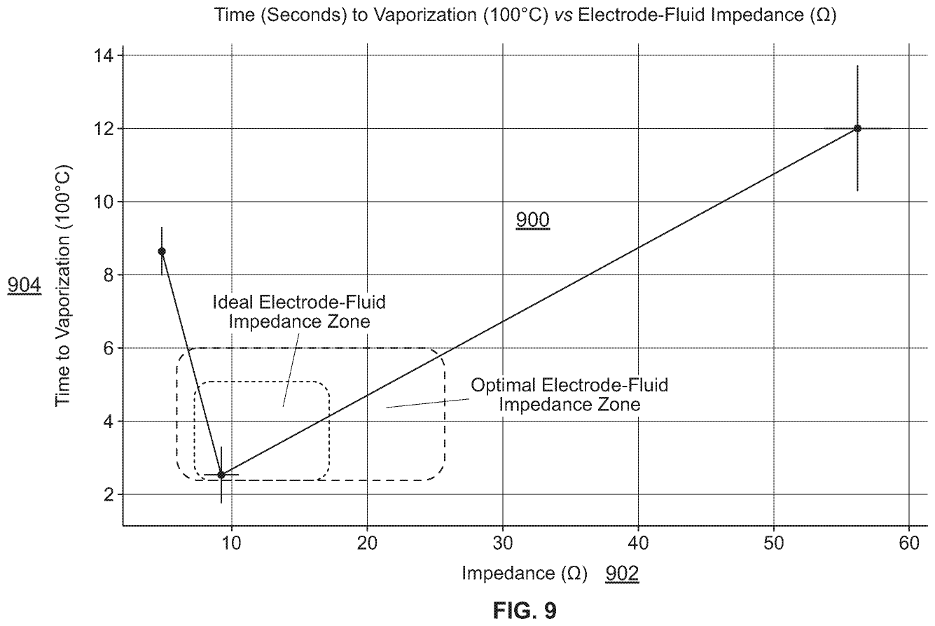

FIG. 9 is a graph 900 illustrating a relationship between electrode-fluid impedance and time to vaporization. Graph 900 demonstrates that vaporization time varies with electrical loading conditions. Referring to FIG. 9, a relationship is shown between electrode-fluid impedance 902 (shown along the horizontal axis) and time to vaporization of a working fluid 904 (shown along the vertical axis), where time to vaporization corresponds to the time required to heat a working fluid from an initial condition to a vaporization condition (approximately 100 °C) under a defined RF heating protocol. In some embodiments, the initial condition corresponds to the working fluid being at delivery temperature, such as room temperature.

FIG. 9 identifies one or more impedance ranges associated with achieving a target vaporization time, including achieving room-temperature fluid-to-vapor conversion in less than about 5 seconds. In some embodiments, the system and the working fluid are co-optimized such that the combined electrode-fluid load impedance is maintained within a target operating region that increases power available at the electrode and reduces time-to-target temperature. In some embodiments, the target operating region is from about 6 Ω to about 30 Ω, and in some embodiments a preferred region is from about 10 Ω to about 25 Ω. In some embodiments, a preferred operating region is centered near about 10 Ω. In some embodiments, an ideal region for a first implementation is about 5 Ω to about 15 Ω, and an ideal region for a second implementation is about 5 Ω to about 30 Ω. In some embodiments, the system is configured to operate across a broader impedance envelope, for example from about 0.5 Ω to about 500 Ω (including about 1 Ω to about 200 Ω), while providing one or more preferred subranges in which delivered power and heating rate are increased. In some embodiments, the controller monitors impedance and/or one or more related electrical parameters (forward power, reflected power, phase) and adjusts one or more delivery parameters (RF power, duty cycle, frequency) and/or one or more fluid parameters to maintain operation within a selected impedance region, subject to safety constraints. In some embodiments, faster heating is preferred.

The systems and methods described herein provide an RF-based vapor generation platform that integrates: coaxial vaporization architecture, porous phase-separation membranes, impedance-aware RF control, fluid recirculation and flushing systems, and optional gas-assisted delivery mechanisms. These features cooperate to improve vapor quality, enhance energy efficiency, and provide stable and controllable vapor delivery for therapeutic procedures.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A catheter-based vapor generation system comprising:
an elongate shaft comprising a fluid delivery lumen longitudinally extending within the elongate shaft, wherein the elongate shaft has a distal end;
at least one electrode positioned proximate to an end of the fluid delivery lumen at the distal end of the elongate shaft and configured to heat a working fluid delivered by the fluid delivery lumen to generate a heated vapor-rich two-phase mixture;
a porous membrane filter completely surrounding the at least one electrode and defining an enclosed annular volume, wherein the porous membrane filter is configured to permit passage of the vapor from the two-phase mixture out of said enclosed annular volume while restricting a release of liquid from the two-phase mixture, thereby maintaining liquid within said enclosed annular volume;
an impedance sensing module configured to measure an electrode-fluid load impedance associated with the at least one electrode; and
a controller configured to:
control delivery of energy to the at least one electrode; and
modify at least one operating parameter of the vapor generation system in response to a detected change in the electrode-fluid load impedance.

2. The system of claim 1, wherein the energy is radio frequency (RF) and wherein the at least one operating parameter is at least one of RF power, RF frequency, RF duty cycle, a rate of the working fluid flow, and a volume of the working fluid flow.

3. The system of claim 1, wherein the porous membrane filter comprises a hydrophobic polymer material adapted to retain said liquid within the annular volume while permitting said vapor to flow out said annular volume.

4. The system of claim 3, wherein the porous membrane filter comprises pore sizes having a size range from 0.1 micrometer (μm) to 1.0 μm.

5. The system of claim 1, wherein the energy is radio frequency (RF) energy and wherein the controller comprises an adaptive RF modulation system that adjusts at least one of RF power and RF frequency based on measured impedance changes at the at least one electrode.

6. The system of claim 1, wherein the annular volume is defined between an inner surface of the porous membrane filter and at least one of an outer surface of the fluid delivery lumen and an outer surface of the at least one electrode.

7. The system of claim 1, wherein the annular volume comprises at least one structural support member disposed between the at least one electrode and the porous membrane filter and configured to maintain a predetermined working fluid layer thickness.

8. The system of claim 1, wherein the at least one electrode comprises a plurality of traces separated from each other by a spacing and wherein the at least one structural support extends perpendicularly from a surface of the at least one electrode and has a height equal to about 0.2 to 10 times the inter-trace distance.

9. The system of claim 1, wherein the controller is configured to trigger a fluid flush cycle in response to a detected impedance of liquid in the annular volume corresponding to an increased salinity of the working fluid.

10. The system of claim 9, wherein the increased salinity is defined by a saline concentration of the liquid in the annular volume exceeding 5%.

11. The system of claim 9, further comprising a fluid return lumen in fluid communication with the annular volume and configured to remove liquid from the annular volume during the fluid flush cycle.

12. The system of claim 11, further comprising a gas delivery lumen, separate from the fluid delivery and the fluid return lumen, configured to introduce a gas into the annular volume such that the gas mixes with generated vapor to form a gas-vapor mixture delivered through the porous membrane filter.

13. The system of claim 12, wherein the gas comprises carbon dioxide and forms a heated gas-vapor mixture with the generated vapor.

14. A method of generating and delivering heated vapor, comprising:
providing a catheter having a distal end comprising at least one electrode, a porous filter circumferentially positioned around the at least one electrode and defining a vaporization region, and a fluid delivery lumen having an end terminating proximate the at least one electrode and the porous filter;
delivering a working fluid through the fluid delivery lumen and into the vaporization region;
applying RF energy to the at least one electrode in the vaporization region to generate a two-phase mixture of vapor and liquid;
permitting the vapor of the two-phase mixture to pass through the porous membrane and out of the vaporization region while concurrently restricting the liquid of the two-phase mixture to pass through the porous membrane and out of the vaporization region;
measuring an electrode-fluid load impedance associated with the at least one electrode;
using a controller, monitoring an electrical parameter indicative of a change in working fluid concentration based on the measured electrode-fluid load impedance;
using the controller, adjusting at least one of RF power, RF duty cycle, RF frequency, and working fluid flow responsive to the monitored electrical parameter; and
performing a flush cycle by introducing fresh working fluid into the vaporization region through the fluid delivery lumen and removing concentrated working fluid in the vaporization region through a return lumen.

15. The method of claim 14, wherein the porous membrane comprises a hydrophobic polymer material adapted to retain said liquid within the vaporization region while permitting said vapor to flow out said vaporization region and comprises pore sizes having a size range from 0.1 micrometer (μm) to 1.0 μm.

16. The method of claim 14, wherein the vaporization region comprises at least one structural support member disposed between the at least one electrode and the porous membrane and configured to maintain a predetermined working fluid layer thickness.

17. The method of claim 16, wherein the working fluid layer thickness is equal to about 0.2 to 10 times an inter-trace distance of the at least one electrode.

18. The method of claim 16, wherein the at least one electrode comprises a plurality of traces separated from each other by a spacing and wherein the at least one structural support extends perpendicularly from a surface of the at least one electrode and has a height equal to about 0.2 to 10 times the inter-trace distance.

19. The method of claim 16, wherein the vaporization region has a thickness equal to about 0.2 to 10 times an inter-trace distance of the at least one electrode.

20. The method of claim 14, wherein the catheter further comprises a gas delivery lumen, separate from the fluid delivery and the return lumen, configured to introduce a gas into the volume such that the gas mixes with generated vapor to form a gas-vapor mixture delivered through the porous membrane.
